(19) United States
(12) Patent Application Publication
(10) Pub. No.: US 2021/0316060 A1
(43) Pub. Date: Oct. 14, 2021

(54) MEDICAL DRAINAGE MONITORING METHOD, APPARATUS, AND SYSTEM BASED ON IMAGE RECOGNITION

(71) Applicant: Tienan LIU, Tangshan (CN)

(72) Inventors: Tienan LIU, Tangshan (CN); Mengchi AI, Tangshan (CN); Chuntao WU, Tangshan (CN); Zhuoran DANG, Tangshan (CN)

(73) Assignee: Tienan LIU, Tangshan (CN)

(21) Appl. No.: 17/304,841

(22) Filed: Jun. 26, 2021

Related U.S. Application Data
(63) Continuation-in-part of application No. PCT/CN2019/126662, filed on Dec. 19, 2019.

(30) Foreign Application Priority Data
Dec. 29, 2018 (CN) 201811652811.4
Dec. 16, 2020 (CN) 202023032821.3

(51) Int. Cl. A61M 1/00

(52) U.S. Cl. CPC A61M 1/69; A61M 1/84; A61M 1/88; A61M 2205/3334; A61M 2205/587; A61M 2205/3379; A61M 2205/18

(57) ABSTRACT

Disclosed are a medical drainage monitoring method, apparatus, system based on image recognition. A medical drainage monitoring system comprises: a drainage device comprising a frame and a drainage container disposed in the frame, the drainage container having an inlet for receiving drained liquid; a first video acquisition device mounted on the frame aimed at the drainage container for obtaining video data of the drained liquid in the drainage container; an image processing circuit, configured to receive and process the video data from the first video acquisition device to obtain first drainage data of the drained liquid; and a monitoring circuit configured to monitor the first drainage data and trigger an alarm signal when the first drainage data violates a predetermined threshold.

### CROSS-REFERENCE TO RELATED APPLICATIONS

The present application claims the priority of Chinese Patent Application No. 201811652811.4 filed on Dec. 29, 2018, and Chinese Patent Application No. 202023032821.3, filed on Dec. 16, 2020, the disclosure of each of the above-mentioned applications are hereby expressly incorporated by reference herein in their entireties.

### TECHNICAL FIELD OF THE DISCLOSURE

The present disclosure generally relates to medical equipment, and in particular, to a drainage monitoring method, apparatus, and system based on image recognition.

### BACKGROUND

The device for draining body fluid, e.g., a drainage bottle or bag, is one of the most common medical equipment in the medical field and is widely utilized to various clinical applications. In the application process of clinical diagnosis and treatment, the drainage device is usually placed at the bottom of the sickbed, and the body fluid transports into the drainage device by means of gravity or negative pressure.

The change of the state of diseases during the drainage process can be characterized through the properties and states of drained liquid that can be measured, such as volume, flow rate, color, turbidity, etc. Specifically, the volume of the drained liquid reflects the accumulation degree of the effusion, blood, pus and other fluids in the body cavity of the patient. The flow rate reflects the internal pressure within the body cavity of the patient. The color and turbidity of the drained liquid reflect the condition of the tissue of the drainage area to a certain extent. Currently, the medical drainage device is monitored by means of manual inspection, which can leads to two main shortcomings. Firstly, the monitoring process of drainage treatment generally needs a long time, e.g., several days or even weeks for some occasions. A lot of manpower would therefore be consumed, and it can be hard to fulfill the need, especially when at night or nursing personnel is in shortage. Secondly, the key properties of drained liquid for the detection of disease state change can be hard to gather accurately through manual visualization. In addition, emergency situation can happen sometimes that the flow rate of drainage liquid suddenly increases in short time or the characteristic of drainage liquid changes when the illness state of the patient deteriorates, such as vascular rupture causing massive hemorrhage, or an abnormal substance doped in the drainage liquid turning the liquid into a turbid state from a clear state, all of which require nursing personnel to find out in time and carry out proper treatment. However, it is difficult to find such changes in time using the conventional mode of manual observation and monitoring, which would delay rescue, thus causing serious consequences and even endangering the lives of patients.

In view of the above, it is desirable to provide a drainage monitoring device that can simultaneously perform dynamical monitoring on the drainage data including volume, flow rate, color and turbidity of the drained liquid, and enable in-time discovery, record, and alarm and treatment plan generation for a drainage abnormality, thereby reducing the workload of nursing personnel, and reducing the medical risk that may be caused by intermittent manual monitoring to the largest extent.

### SUMMARY

In order to solve at least one of the above technical problems, the present disclosure provides a medical drainage monitoring method, apparatus, system, which can monitor important information in the drainage process such as the volume, flow rate, color and turbidity of the drained liquid and whether bubbles exist in the drained liquid or not, and the like, effectively gather and record relevant data, and activate an alarm when an abnormality occurs.

According to one aspect of the present disclosure, a medical drainage monitoring system is provided, which may comprise: a drainage device comprising a frame and a drainage container disposed in the frame, the drainage container having an inlet for receiving drained liquid; a first video acquisition device mounted on the frame and aimed at the drainage container for obtaining video data of the drained liquid in the drainage container; an image processing circuit, configured to receive and process the video data from the first video acquisition device to obtain first drainage data of the drained liquid; and a monitoring circuit configured to monitor the first drainage data and trigger an alarm signal when the first drainage data violates a predetermined threshold.

As compared with the prior art, the drainage monitoring system according to the embodiment of the present disclosure can monitor various drainage information such as the volume, flow rate, color and turbidity of the drained liquid and whether bubbles exist in the drained liquid or not, and the like, based on an image processing. The drainage monitoring system can enable processing, recording of the above-mentioned drainage data and triggering an alert in case of abnormality, and thus reduce the workload of nursing personnel. The drainage monitoring system may be applied to various clinical occasions, which greatly reduces medical risks.

### BRIEF DESCRIPTION OF THE DRAWINGS

Through a more detailed description of the embodiments of the present disclosure in conjunction with the accompanying drawings, the above and other objectives, features and advantages of the embodiments of the present disclosure will become more apparent. The drawings are included to provide a further understanding of the embodiments of the present disclosure, and are incorporated in and constitute a part of the specification to explain the present disclosure together with the embodiments of the present disclosure, but are not intended to serve as a definition of the limits of the present disclosure. In the drawings, the same reference numeral usually represents an identical component.

FIG. 1 shows a schematic diagram of a frame for receiving a drainage bottle according to an embodiment of the present disclosure.

FIG. 2 shows a schematic side view of a frame for receiving a drainage bottle according to an embodiment of the present disclosure.

FIG. 3 shows a schematic diagram of a drainage bottle according to an embodiment of the present disclosure.

FIG. 4 shows a schematic diagram of a drainage device according to an embodiment of the present disclosure, in which the drainage device includes a frame and a drainage bottle received in the frame.

FIG. 5 is a schematic diagram of a drainage bag that may be received in the frame of FIG. 1 according to an embodiment of the present disclosure.

FIG. 6 is a block diagram of a medical drainage monitoring system according to an embodiment of the present disclosure.

FIG. 7 is a block diagram of an image processing circuit according to an embodiment of the present disclosure.

FIG. 8 is a block diagram of an image processing circuit according to another embodiment of the present disclosure.

FIG. 9 is a block diagram of a monitoring circuit according to an embodiment of the present disclosure.

FIG. 10 is a block diagram of a central monitoring device according to an embodiment of the present disclosure.

FIG. 11 is a flowchart of a drainage monitoring method according to an embodiment of the present disclosure.

### DETAILED DESCRIPTION

Hereinafter, exemplary embodiments according to the present disclosure will be described in detail with reference to the drawings. It should be understood that the specific embodiments described herein are only used to explain the related disclosure, but are not intended to limit the disclosure.

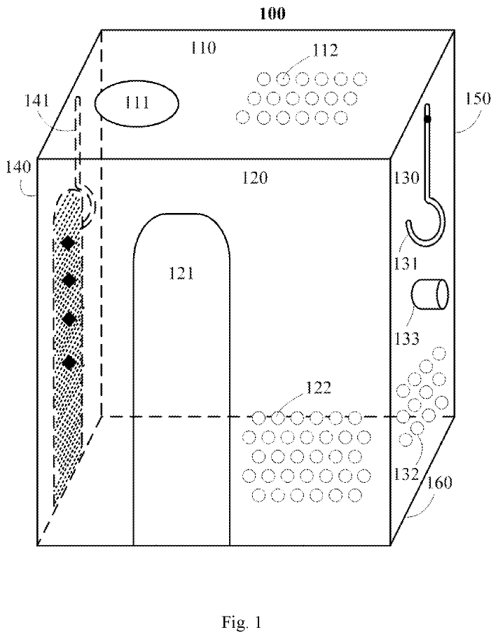

An embodiment of the present disclosure provides a drainage device including a drainage container and a frame for receiving the drainage container. FIG. 1 shows a schematic diagram of a frame 100 for receiving a drainage container. As shown in FIG. 1, the frame 100 may have a cubic shape, and may include a cover plate 110, side walls 120, 130, 140, and 150, and a bottom wall 160, which surround an interior cavity that can receive the drainage container.

The cover plate 110 may be connected to an upper edge portion of one of the side walls 120-150 by, for example, a hinge (not shown), so that the cover plate 110 may be opened and closed. Alternatively, the cover plate 110 may be detachably mounted to the upper edge portion of the side walls 120-150. Additionally, a snap joint may also be provided to fix the cover plate 110 to the upper edge of the side wall. One or more openings 111 may be provided on the cover plate 110 for connecting an inlet interface of the drainage container to the outside through the opening(s). Optionally, one or more venting holes may be provided on the cover plate 110, such as an array of venting holes 112 shown in FIG. 1, which can facilitate heat dissipation. In some embodiments, the cover plate 110 may be omitted, and the entire upper surface of the frame 100 is in an open state.

The first side wall 120 may be formed as the front surface of the frame 100 with an observation window 121 provided thereon. The observation window 121 may extend in an up-down direction, for example, and is used to manually observe the drainage container received in the frame 100, which will be described in detail later. The observation window 121 may be an opening formed on the first side wall 120 or may be a portion of the first side wall 120 formed of a transparent material. In some embodiments, the entire first side wall 120 may be formed of a transparent material such as glass or plastic, so that the entire first side wall 120 may be used as an observation window 121. It shall be understood that the entire frame 100 or a major part of the frame 100 may also be formed of a transparent material. As shown in FIG. 1, an array of venting holes 122 may also be formed on the first side wall 120.

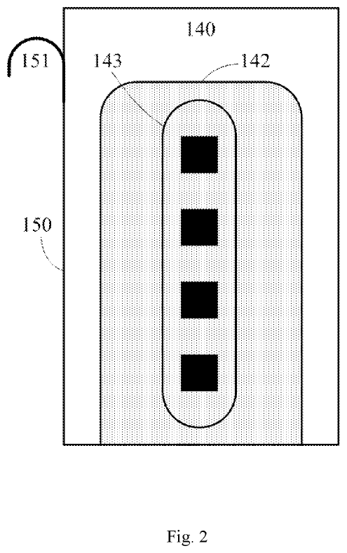

Adjacent to the first side wall 120 are a second side wall 130 and a third side wall 140 opposite to each other, which may be formed with suspension members such as hooks 131 and 141, respectively, for suspending the frame 100 on a support such as a bed head panel, a bed end panel, or a bed edge panel of a hospital bed. It shall be understood that the suspension members are not limited to the hooks 131 and 141 as shown in the figure, but may also adopt other structures, such as a clip-type hook 151 provided on the back side 150 as shown in FIG. 2, which can firmly clamp the frame 100 on a bed head panel, a bed end panel, or a bed edge panel. Other structures such as a lanyard, a sling, and the like, may also be used. In some embodiments, the suspension member may also be omitted, and the frame 100 may be directly placed on the ground under the hospital bed, for example, or the frame 100 may be directly fixed to the hospital bed through a fastening structure such as a buckle which may be provided on the hospital bed. An array of venting holes 132 may also be formed on the second side wall 130. It shall be understood that the shape, size, number, arrangement of the venting holes on the frame 100 are not limited to those shown in FIG. 1. The second side wall 130 is also formed with one or more sensor mounting portions 133, which may be located in the middle of the second side wall 130. The sensor mounting portion may be a circular hole formed in the second side wall 130, or may include a protruding structure with a central circular hole protruding from the second side wall 130. The circular hole may be connected with the interior cavity of the frame 100. Examples of the sensor(s) may include a camera, a laser sensor, and/or an ultrasonic sensor, etc., which may be mounted into the circular hole of the sensor mounting portion 133 through interference fit, adhesive bonding, or thread connection, and the like. For example, a camera may be mounted in the circular hole of the sensor mounting portion 133 to capture an image of the interior cavity of the frame 100.

A diffusion portion 142 may be formed on the third side wall 140, as shown in the side view of the frame. As illustrated in FIG. 2, the diffusion portion 142 may be formed by forming an uneven structure on the inner wall or the outer wall of the third side wall 140, or may be a diffusion film attached to the third side wall 140. A light source 143 may be provided on the outer wall of the diffusion portion 142, which may include, for example, a plurality of LED elements. The light emitted by the light source 143 is diffused by the diffusion portion 142 to generate a soft light to illuminate the inside of the frame 100. Although not shown, the outside of the light source 143 may be provided with a light blocking portion or a light reflecting portion to avoid the influence of the light source on the external environment and ensure illumination brightness to the inside of the frame 100. In addition, a battery mounting portion may also be formed on the frame 100 for mounting a battery for supplying power to the light source 143. In some embodiments, the light source 143 may also be connected to a socket through a cable plug to obtain electrical power. It shall be understood that the light source 143 and the diffusion portion 142 may also be formed on other side walls of the frame 100, and not limited to the side wall 140 opposite to the camera mounting portion 133. FIG. 2 also shows a back hook 151 is formed on the fourth side wall 150, and it may be used to suspend the frame 100 on a support such as a bed end panel or a bed edge panel.

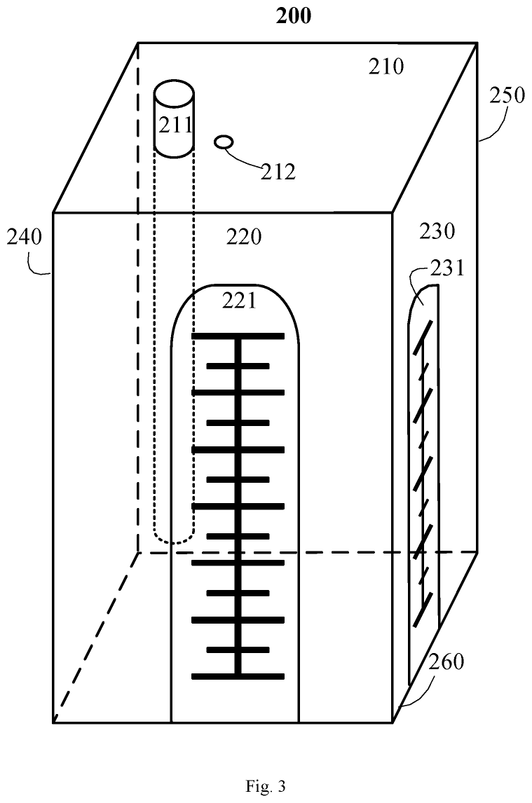

In some embodiments, the drainage container that is received in the frame 100 shown in FIGS. 1-2 may include a drainage bottle or a drainage bag. FIG. 3 shows a schematic diagram of a drainage bottle 200 according to an embodiment of the present disclosure. As shown in FIG. 3, the drainage bottle 200 has a cubic structure, and includes a top wall 210, side walls 220, 230, 240, and 250, and a bottom wall 260, which surround an internal cavity that can receive the drained liquid. When the drainage bottle 200 is placed within the frame 100, each wall 210-260 of the drainage bottle 200 corresponds to each wall 110-160 of the frame 100.

A plurality of interfaces may be formed on the top wall 210 of the drainage bottle 200, such as a drained liquid interface 211 for the drained liquid to flow in and a venting hole 212 for exhausting gas. When the drainage bottle 200 is placed in the frame 100, the drained liquid inlet interface 211 may protrude from the opening 111 on the cover plate 110 of the frame 100 so as to connect the drainage tube to the drained liquid interface 211.

A first volume indicator 221 for indicating the volume of the drained liquid may be formed on the first side wall 220. The first volume indicator 221 may be a transparent portion, and may include a dark volume scale line, so as to observe the amount of the drained liquid received in the drainage bottle 200. A second volume indicator 231 may be formed on the second side wall 230, which may also be a transparent portion, and may include a dark volume scale line, so as to observe the amount of the drained liquid received in the drainage bottle 200. It shall be understood that, in some embodiments, the entire drainage bottle 200 may be formed of a transparent material such as plastic, glass, and the like.

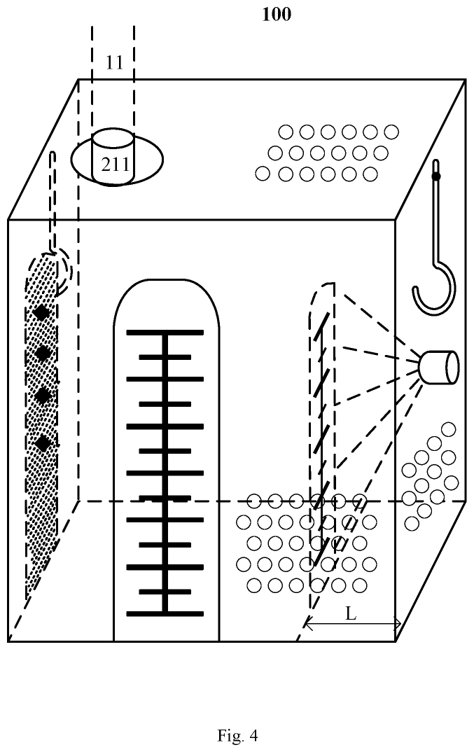

FIG. 4 shows a schematic diagram of a drainage device 10 in which the drainage bottle 200 is placed in the frame 100. It shall be understood that a guide rail may be provided in the inner wall of the frame 100 to guide the drainage bottle 200 to be placed in a predetermined position in the frame 100, and to fix the drainage bottle 200 so as to avoid shaking within the frame 100. As shown in FIG. 4, when the drainage bottle 200 is placed in the frame 100, the drained liquid interface 211 of the drainage bottle 200 is exposed to the outside through the opening 111 in the cover plate 110 of the frame 100, and the drainage tube 11 (as shown by the dashed lines) may be connected to the drained liquid interface 211. The drainage bottle 200 may be close to the side wall 140 provided with the diffusion portion 142, so that the light emitted by the light source 143 is irradiated onto the drainage bottle 200 after being diffused. The first volume indicator 221 on the drainage bottle 200 may be exposed through the opening 121 on the front surface of the frame 100 so as to facilitate manually observing drainage data such as the volume and color of the drained liquid received in the drainage bottle 200. In addition, the camera mounted in the sensor mounting portion 133 on the frame 100 may acquire an image of the second volume indicator 231 of the drainage bottle 200, and then the drainage data such as the volume, color and flow rate of the drained liquid in the drainage bottle 200 may be determined through image processing, so that automatic machine monitoring of the drainage process may be realized, and the labor burden of nursing personnel is reduced. The camera may send the acquired image to the image processing device in a wired or wireless manner.

In order to facilitate the camera to acquire images, as shown in FIG. 4, there may exist a distance L between the drainage bottle 200 and the camera, or between the drainage bottle 200 and the side wall 130 of the frame 100 for mounting the camera. In addition, the camera may use a wide-angle camera, so that the second volume indicator 231 may be fully and clearly imaged. The size of L may be determined according to the imaging parameters of the camera, and generally, may be in the range of 3 cm to 40 cm, preferably in the range of 4 cm to 30 cm, and more preferably in the range of 5 cm to 20 cm. It shall be understood that the frame 100 in the drainage device 10 may be reused, and the drainage bottle 200 may be easily replaced, thereby reducing the cost.

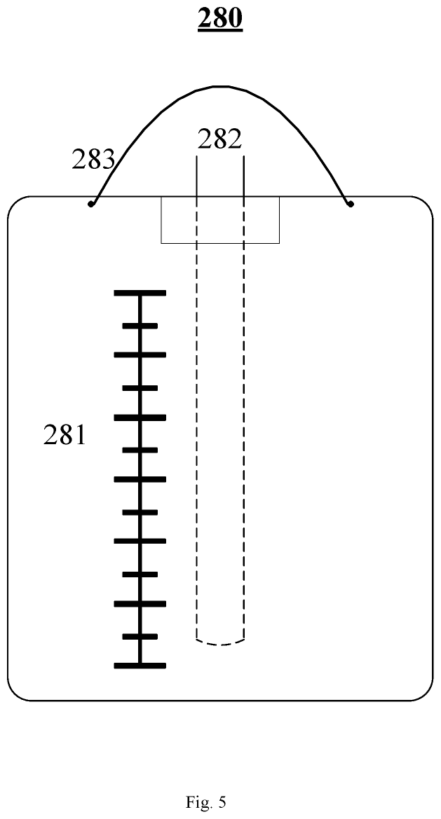

FIG. 5 shows a schematic diagram of a drainage bag 280 that may be received in the frame 100 shown in FIGS. 1-2 according to an embodiment of the present disclosure. As shown in FIG. 5, the drainage bag 280 may have a generally rectangular shape, or may have other shapes. A volume scale line 281 may be formed on one side of the drainage bag 280. When the drainage bag 280 is placed in the frame 100, the volume scale line 281 may face the sensor mounting portion 133 so as to obtain information such as the volume of the drained liquid. The upper side of the drainage bag 280 may include an inlet 282 for receiving the drained liquid, or may further include an exhaust hole (not shown) for exhausting or vacuuming. The upper side of the drainage bag 280 may also be provided with a suspension device such as a lanyard 283 shown in FIG. 5. The suspension device may also be, for example, a suspension hole formed in the drainage bag 280. When the drainage bag 280 is placed in the frame 100, the lanyard or suspension hole 283 may be suspended on a hook (not shown) formed on the inner wall of the frame 100. The drainage bag 280 may have, for example, a structure with two or more layers, which may be formed of a transparent plastic material.

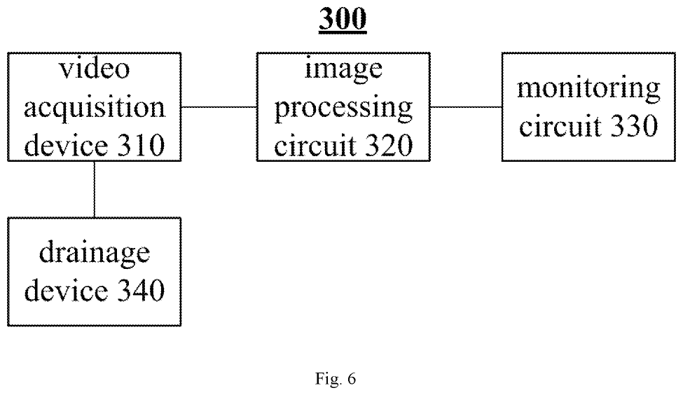

FIG. 6 is a block diagram of a drainage monitoring system 300 according to an embodiment of the present disclosure. As shown in FIG. 6, the drainage monitoring system 300 may comprise: a video acquisition device 310, which can capture an image of the drained liquid and obtain the video streaming data of the drained liquid; an image processing circuit 320, which is connected with the video acquisition device 310, and can receive the video streaming data and process the video streaming data to obtain drainage data of the drained liquid; a monitoring circuit 330, which is connected with the image processing circuit 320, and is used to receive and monitor the drainage data; and a drainage device 340, which may be implemented as, for example, the drainage device 10 as described above with reference to FIGS. 1-5. In one example, the drainage device includes a frame 100 and a drainage container such as a drainage bottle 200 or a drainage bag 280, and can receive drained liquid from the patient through the catheter 11. For the convenience of description, the drainage bottle 200 is taken as an example below to illustrate some embodiments, but it should be understood that the drainage bag 280 can also be used instead of the drainage bottle 200.

The video acquisition device 310 may include one or more cameras, which may continuously acquire multiple frame images to form video streaming data, so as to realize dynamic real-time monitoring of the drainage process. In one embodiment, the video acquisition device 310 includes a camera mounted at the sensor mounting portion 133 of the frame 100 as shown in FIG. 1, which is aimed at the drainage bottle 200, so as to capture an image of the drainage bottle 200 and the drained liquid received therein. As mentioned above, the side 230 of the drainage bottle 200 is provided with a volume scale line 231, and the current drained liquid volume may be determined by identifying the position of the liquid level of the drained liquid on the volume scale line 231. In addition, the current drainage flow rate may be calculated by monitoring the change of the drained liquid volume over time. In addition, the camera aimed at the drainage bottle 200 can also observe whether bubbles exist in the drained liquid which is in the drainage bottle 200 or whether bubbles exist on the liquid level of the drained liquid, which is also one of the important pieces of information that needs to be monitored during the medical drainage process.

A very important task in the drainage process is to monitor whether the patient has abnormal bleeding symptoms, which may be determined by monitoring the color of the drained liquid. The inventor of the present disclosure found that, in a case where the drained liquid enters the drainage bottle 200 from the drainage tube, the color thereof may be diluted by the large amount of drained liquid already received in the drainage bottle 200. Therefore, the bleeding symptoms may not be detected in time, which will delay the rescue opportunity and cause serious medical consequences. In an embodiment of the present disclosure, the video acquisition device 310 may further include a second camera configured to be aimed at the drainage tube, which is used to monitor the color information of the drained liquid in the drainage tube, so as to monitor more timely whether blood is included in the drained liquid. The video acquisition device 310 for monitoring the drainage tube may be mounted on the support and be aimed at the drainage tube 11 (as shown in FIG. 4). At least a portion of the drainage tube 11, such as the portion monitored by the video acquisition device 310, is made of a colorless and transparent material such as glass or plastic.

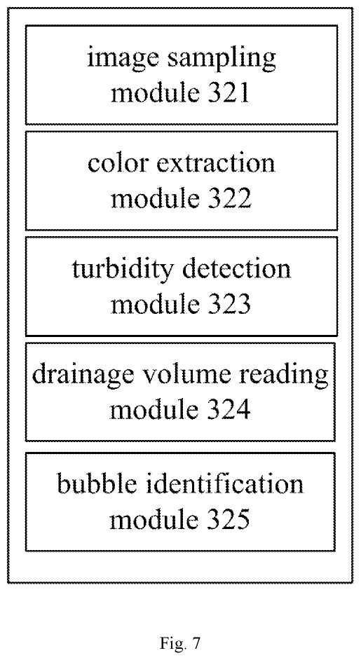

The image processing circuit 320 can receive the video data acquired by the video acquisition device 310 such as one or more cameras, and determine drainage information such as volume, flow rate, color, turbidity of the drained liquid and whether bubbles exist in the drained liquid through image recognition. FIG. 7 is a block diagram of an image processing circuit 320 according to an embodiment of the present disclosure. As shown in FIG. 7, the image processing circuit 320 may include an image sampling module 321, a color extraction module 322, a turbidity detection module 323, a volume and flow rate determination module 324, and a bubble identification module 325.

The video data acquired by each camera may include a plurality of image frames arranged in temporal order, and the number of image frames included per second depends on the performance of the camera itself. Generally, it is not necessary to process each frame of images in the video data because the difference between adjacent image frames may not be obvious, otherwise it may result in heavy computational burden and high hardware requirements. Therefore, the image processing circuit 320 may include an image sampling module 321 to sample the video data acquired by the camera to extract image frames. For example, the image sampling module 321 may extract image frames from the video data at a fixed sampling rate, for example, by extracting 1 frame image every 0.5 seconds. In some embodiments, the image sampling module 321 can dynamically adjust the sampling rate according to the differentiation of the sampled image frames. For example, the image sampling module 321 may compare the currently sampled frame image with the previously sampled frame image. If the sampled image embodies change information at a high level, the sampling rate may be increased; if the sampled image embodies change information at a low level, the sampling rate may be reduced; and if the sampled image does not change substantially, the multiple frame images may be sampled at a predetermined minimum rate, for example, at one frame per second. Through such dynamic sampling, the changes or variations in the drainage process may be monitored more finely, while avoiding the generation of large amounts of sampling data.

The color extraction module 322 can extract the color information of the drained liquid by performing image recognition on the sampled image frames. As mentioned above, preferably, the color extraction module 322 can perform image recognition on one or more image frames obtained from the camera aimed at the drainage tube to recognize the drainage tube portion and extract the color of the drained liquid in the drainage tube, so that the color change of drained liquid may be timely and accurately recognized, and the effect of dilution to the color by the drained liquid received in the drainage bottle 200 may be mitigated. In another example, the color extraction module 322 may also perform image recognition on the image frame of the drainage bottle 200 to extract the color of the drained liquid received therein. The color extraction module 322 may recognize an RGB value of a plurality of pixel points representing the drained liquid in the image of drainage tube or drainage bottle, and calculate an average value thereof to determine the color of the drained liquid in the image frame. In some embodiments, the color of the drained liquid in the image frame may be interfered due to various factors such as the light condition and the transmittance of the material forming the drainage bottle or the drainage tube. To counter the interference, in one embodiment, the color extraction module 322 may also correct the recognized color. For example, a standard color card may be placed at or near the drainage bottle 200 or the drainage tube 11, and an image of the standard color card may be captured by the video acquisition device 310. After the color of the standard color card is extracted and recognized by the color extraction module 322, the recognized color and the genuine color are compared to obtain a deviation value between the two, which may be used as a correction factor to correct the color recognized by the color extraction module 322.

In an embodiment, the image sampling module 321 may be configured to adjust the sampling rate based on differentiation in color information. For example, when the color differentiation of any channel in the RGB color values of the drained liquid recognized by the color extraction module 322 reaches a predetermined threshold, or the sum of the color differentiation of a plurality of channels reaches a predetermined threshold, the image sampling module 321 may increase the sampling rate. For example, the sampling rate may be adjusted from 2 image frames per second to 10 image frames per second, which makes the monitoring of any variation in the drainage process more accurate.

The turbidity detection module 323 is used for processing the image frames and determining the turbidity information of the drained liquid. In one embodiment, the brightness information of the image is used to characterize the turbidity of the drained liquid. The turbidity detection module may extract the brightness value of the drained liquid from the image frame, and determine the turbidity of the drained liquid based on the extracted brightness value. Various methods may be used to extract the brightness value of the drained liquid pixels in the image frame. For example, the color value of the drained liquid pixels may be converted to the YUV space from the RGB space, and the Y value may be used as the turbidity of the drained liquid, which can be presented as a conversion formula Y=0.299*R+0.587*G+0.114*B. In one embodiment, the color values of the drained liquid pixels may also be converted from RGB space to other color spaces, such as LAB space or HSI space, and the L component or the I component can be used as the brightness value of the drained liquid. In some embodiments, the average value or the mean square value of the brightness values of a plurality of drained liquid pixels may be calculated as the turbidity of the drained liquid. If the turbidity information (brightness value) of the drained liquid is greater than or equal to a threshold, it indicates that the drained liquid is in a clear state; and if the brightness value of the drained liquid is lower than the threshold, it indicates that the drained liquid is in a turbid state. For example, in a case where the drained liquid includes pus, blood clots and other components, then it will be in a turbid state.

The volume and flow rate determination module 324 may be used to determine the drained liquid volume and flow rate information by performing image recognition on the image frames. For example, as described above with respect to FIGS. 1-4, the volume of the drainage fluid may be determined by recognizing the position of the liquid level of the drained liquid in the volume scale line 211; the drainage flow rate may be determined by identifying the changes in the drained liquid volume between different frames. In one embodiment, the image frame may be converted to a grayscale mode, followed with a median filtering processing on the image frame to enhance the liquid level line in the image. Then an edge enhancement processing may be performed on the processed image, so that the liquid level line of the drained liquid is extracted. Consequently, the volume of the drainage fluid may be determined based on the position of the liquid level line in the volume scale line 211.

The bubble identification module 325 may recognize bubbles in the sampled image frame by using image recognition technology. The bubbles may be present in the drained liquid when discharged from the drainage tube, or may float on the surface of the drained liquid. Since the bubbles have a substantially circular shape boundary, it is possible to recognize whether there are bubbles in the drained liquid or on the surface of the drained liquid through the image recognition technology.

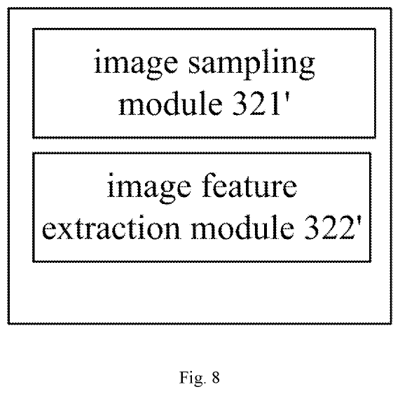

In another embodiment, a machine learning method may be used to extract drained liquid information such as color and turbidity in the image frame. As shown in FIG. 8, the image processing circuit 320 may include: an image sampling module 321′ and an image feature extraction module 322′, where the function and operation of the image sampling module 321′ are the same as the previously described image sampling module 321, and will not be repeated here.

The image feature extraction module 322′ is a neural network-based model for extracting drainage data related to the drained liquid from image frames. In one embodiment, the image feature extraction module 322′ may use a convolutional neural network (CNN) model, using the image data in the YUV color space as an input, and use the genuine value of the drainage data related to the drained liquid as an output to train the CNN model. The model obtained through training may be used for detecting the drainage data related to the drained liquid in the image frame obtained by the image sampling module 321′, including information such as color, turbidity, volume, flow rate, and whether bubbles exist or not. In particular, the neural network model may determine the volume of the drained liquid by recognizing the position of the liquid level in each frame of images, and determine the flow rate of the drained liquid based on the change in the drained liquid volume between adjacent image frames. The information of color, turbidity and bubble of the drained liquid may be determined by recognizing each frame of images.

As mentioned above, the video acquisition device 310 may include a plurality of cameras, such as a first camera aimed at the drainage bottle 200 and a second camera aimed at the drainage tube 11. In some embodiments of the present disclosure, information such as the drained liquid volume and flow rate and bubbles may be determined based on one or more images of the drainage bottle 200 acquired by the first camera, and the color and turbidity of the drained liquid are determined based on the image of the drainage tube 11 acquired by the second camera. In this way, changes of the color and turbidity of the drained liquid in the drainage process may be monitored more accurately.

Continuing to refer to FIG. 6, the image processing circuit 320 may provide the determined drained liquid information, such as volume, flow rate, color, turbidity, and the like, to the monitoring circuit 330. The monitoring circuit 330 may compare the received drainage information with a set value to monitor whether the drainage process is abnormal, as described in detail below.

In an embodiment, the monitoring circuit 330 may monitor the drained liquid volume in the drainage bottle 200. In a case where the drained liquid volume exceeds a predetermined threshold, for example, if an excessive amount of drained liquid is discharged from the body of the patient, or in a case where the drained liquid is to be full of the entire volume of the drainage bottle 200, it can be determined that an abnormality has occurred or is about to occur. Then an alarm may be initiated, so that the patient's family member or nursing staff may be reminded to check or deal with it.

In one embodiment, the monitoring circuit 330 may monitor the color of the drained liquid. For example, in a case where the drained liquid is expected to be colorless liquid, but the monitored color of the drained liquid has color such as red or yellow, which requires timely treatment. In this occasion, the monitoring circuit 330 may initiate an alarm to remind the patient's family member or nursing staff to check or deal with it.

In one embodiment, the monitoring circuit 330 can monitor the turbidity of the drained liquid. For example, in a case where the drained liquid is expected to be clear liquid, but the monitored turbidity of the drained liquid exceeds a predetermined threshold, it may be determined that the drained liquid may include foreign matters such as blood clots, pus, and the like. In this occasion, the monitoring circuit 330 may initiate an alarm to remind the patient's family member or nursing staff to check or deal with it.

In one embodiment, the monitoring circuit 330 may monitor the flow rate of the drained liquid, i.e. the volume of the drained liquid introduced into the drainage bottle 200 per second. Under normal circumstances, the flow rate should be within a reasonable range; if the flow rate is too high, there may be abnormal phenomena such as bleeding occurring in the patient's body; and if the flow rate is too low, the drainage tube may be blocked by foreign matters such as blood clots. Therefore, in a case where the drainage flow rate is outside the reasonable range, it may be determined that an abnormality has occurred, and the monitoring circuit 330 may initiate an alarm, so as to remind the patient's family member or nursing staff to check or deal with it.

In one embodiment, the monitoring circuit 330 may also monitor whether bubbles are contained in the drained liquid. In a case where bubbles are contained in the drained liquid, it may be determined that an abnormality has occurred, and the monitoring circuit 330 can initiate an alarm, so as to promptly remind the patient's family member or nursing staff to check or deal with it.

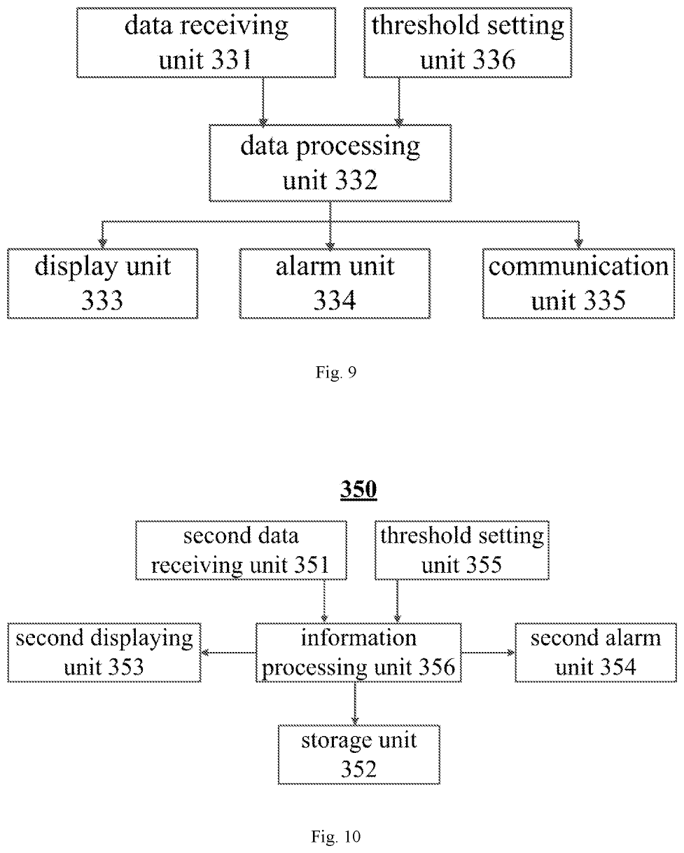

In an example, as shown in FIG. 9, the monitoring circuit 330 includes a data receiving unit 331, a data processing unit 332 and a display unit 333. The data receiving unit 331 may be connected with the image processing circuit 320 to receive drainage monitoring data from the image processing circuit 320, such as the color, turbidity, volume and flow rate of the drained liquid, whether bubbles exist, and the like, which may be encoded in a predetermined format. The data receiving unit 331 may transmit the received data to the data processing unit 332, and the data processing unit 332 may process the received data, such as decoding to extract a color value, a turbidity value, a volume value and a flow rate value of the drained liquid, or whether there is an indicator of bubbles, and the like. These extracted values indicate a drainage state, which may be displayed on the display unit 333. The data processing unit 332 may also compare the drainage data with a predetermined threshold to identify abnormal phenomena, as described above. The predetermined threshold may be set by, for example, a nursing staff through a threshold setting unit 336. In a case where the data processing unit 332 determines that an abnormal drainage may have occurred or is about to occur by comparing the drainage data with a predetermined threshold, the data processing unit 332 may trigger the alarm unit 334 to issue an alarm to remind the patient's family member or nursing staff to check or deal with it. The alarm unit 334 may include, for example, a buzzer and/or a flashing light, and the like.

The monitoring circuit 330 may further include a communication unit 335, which may send relevant data and signals, such as drainage data, alarm signals, and the like, to other devices. For example, the monitoring circuit 330 shown in FIG. 9 may be disposed in a ward. Such monitoring circuit 330 is provided for each patient to monitor the drainage process of the patient. In addition, a central monitoring module may also be provided, for example, in a nurse station or a monitoring management center of a hospital, which may receive data from a plurality of monitoring circuits 330 to monitor the drainage process of a plurality of patients. FIG. 10 shows an example of such a central monitoring module 350, which may be a portion of the drainage monitoring system 300 shown in FIG. 5. The data processing unit 332 of a plurality of monitoring circuits 330 may send the drainage data and related patient information (name, bed number, age, etc.) to the central monitoring module 350 through the communication unit 335. The central monitoring module 350 may include: a data receiving unit 351, which is used for receiving monitoring data sent by a plurality of monitoring circuits 330; a storage unit 352, which is used to store the received monitoring data; and a display unit 353, which may simultaneously display monitoring data of the plurality of monitoring circuits 330, such as patient information and drainage state data, and the like. In some embodiments, the display unit 353 may display data such as a diagram of drained liquid volume, flow rate, color and/or turbidity that change over time in the form of a diagram. Based on these functional modules, the central monitoring module 350 can centrally monitor the drainage process of a plurality of patients and further save the labor cost.

In one example, the central monitoring module 350 may further include: the information processing unit 356, which is configured to process the received patient information and drainage data, such as decoding the received data, and transmitting the decoded data to the display unit 353 for display and to the storage unit 352 for storage. In a case where an alarm signal is contained in the received drainage data, the information processing unit 356 may also trigger the alarm unit 354 to alarm. The information processing unit 356 may also compare the drainage data with a predetermined threshold, which may be set by the threshold setting unit 355, or may be received from the monitoring circuit 330, so as to trigger the alarm unit 354 to issue an alarm when an abnormal phenomenon is found. That is to say, the information processing unit 356 may trigger an alarm in response to an alarm signal received from the monitoring circuit 330, or may actively trigger an alarm by monitoring the drainage data, so as to avoid medical accidents caused by failing to successfully issue an alarm at the monitoring circuit 330.

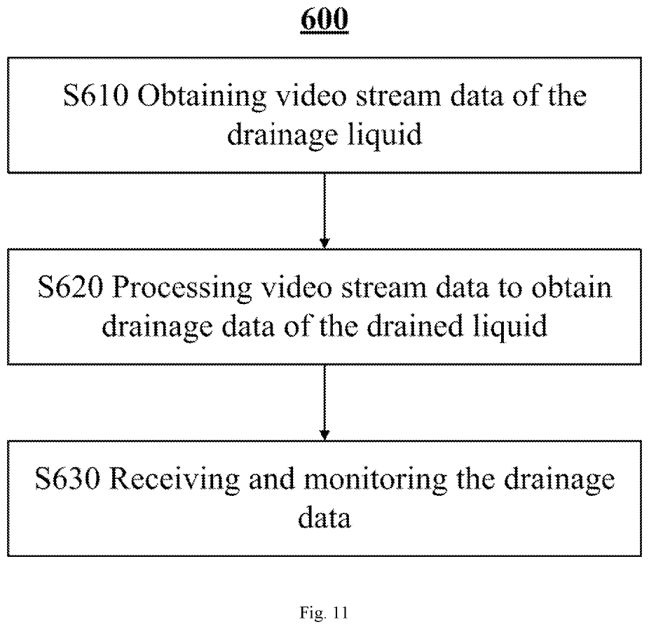

FIG. 11 is a flowchart of a drainage monitoring method 600 according to an embodiment of the present disclosure. Referring to FIG. 11, in step S610, video data of the drained liquid is obtained. As mentioned above, one or more cameras may be used to obtain one or more video data streams, such as video data of the drainage bottle 200 and video data of the drainage tube 11, which may be common formats such as AVI, MPG, and the like.

In step S620, the video data is processed to obtain drainage data. The processing of the video data may be implemented in a data processor with image processing capabilities such as a microcontroller chip or a DSP processor. The obtained drainage data includes the volume, flow rate, color, turbidity, and the like, of the drained liquid. The processing of the video data may include sampling, image recognition, color extraction, turbidity recognition, volume identification, flow rate calculation, and so on, and the specific processing thereof may be implemented by the image processing module as described above, which will not be repeated here.

In step S630, the drainage data is received and monitored. The drainage data may be monitored by a single-chip microcomputer, a processor and the like with data operation and processing capability. For example, the drainage data may be graphically displayed on the screen. The drainage data may also be compared with corresponding threshold to recognize abnormal phenomena, and trigger an alarm in a case where an abnormal drainage occurs. Through the monitoring and alarm functions, the workload of the patients' family member and nursing personnel may be relieved, the medical risk is reduced to the maximum extent. The cost of the system is low, thus producing good economic benefits.

The above description has been presented for the purposes of illustration and explanation. Furthermore, this description is not intended to limit the embodiments of the present disclosure to the form disclosed herein. A person skilled in the art can make various changes and modifications in form and details without departing from the scope and spirit of the present disclosure. That is to say, the scope of the present disclosure is defined by the appended claims and their equivalents.

What is claimed is:

1. A medical drainage monitoring system, comprising:
a drainage device comprising a frame and a drainage container disposed in the frame, the drainage container having an inlet for receiving drained liquid;
a first video acquisition device mounted on the frame and aimed at the drainage container for obtaining video data of the drained liquid in the drainage container;
an image processing circuit configured to receive and process the video data from the first video acquisition device to obtain first drainage data of the drained liquid; and
a monitoring circuit configured to monitor the first drainage data and trigger an alarm signal when the first drainage data violates a predetermined threshold.

2. The medical drainage monitoring system of claim 1, wherein the frame includes:
a plurality of side walls surrounding an interior cavity for receiving the drainage container; and
a video acquisition device mounting portion provided on a first side wall of the plurality of side walls for mounting the first video acquisition device.

3. The medical drainage monitoring system of claim 2, wherein the drainage container comprises a drainage bottle or a drainage bag having a first volume indicator formed thereon, and the video acquisition device mounting portion aligns to the first volume indicator with a first distance therebetween.

4. The medical drainage monitoring system of claim 3, wherein the first distance is in a range of 3-40 cm.

5. The medical drainage monitoring system of claim 3, wherein the frame further includes:
a diffusing portion provided on a second side wall of the plurality of side walls; and
a light source provided on an outer surface of the diffusing portion for emitting light that is diffused by the diffusing portion and irradiated onto the drainage container received in the frame.

6. The medical drainage monitoring system of claim 3, wherein the drainage bottle further includes a second volume indicator provided on a side wall different from the first volume indicator, and the frame further includes an observation window provided in a third side wall of the frame, the second volume indicator being visible through the observation window.

7. The medical drainage monitoring system of claim 2, wherein the frame further includes:
a cover plate for covering the drainage container received in the frame, the cover plate having at least one opening which exposes the inlet of the drainage container.

8. The medical drainage monitoring system of claim 7, wherein the frame further includes a plurality of venting holes provided in one or more of the plurality of side walls and the cover plate.

9. The medical drainage monitoring system of claim 1, wherein the first drainage data includes one or more of volume, flow rate, and color of the drained liquid and information of if there are bubbles in or floating on the drained liquid.

10. The medical drainage monitoring system of claim 1, further comprising:
a second video acquisition device aimed at a drainage tube to obtain video data of drained liquid in the drainage tube, the drainage tube being connected to the inlet of the drainage container,
wherein the image processing circuit is further configured to process the video data from the second video acquisition device to obtain second drainage data of the drained liquid.

11. The medical drainage monitoring system of claim 10, wherein the second drainage data includes color and/or turbidity of the drained liquid.

12. The medical drainage monitoring system of claim 1, wherein the image processing circuit includes one or more of:
an image sampling module for sampling image frames from the video data;
a color extraction module for extracting a color of the drained liquid from the sampled image frames;
a turbidity detection module for detecting a turbidity of the drained liquid from the sampled image frames;
a volume and flow rate determination module for determining a volume of the drained liquid from the sampled image frames, and determining a flow rate of the drained liquid based on a change of the volume over time; and
a bubble identification module for determining from the sampled image frames if there is a bubble in the drainage container.

13. The medical drainage monitoring system of claim 12, wherein the image sampling module is configured to adjust a sampling rate based on differentiation of the sampled image frames.

14. The medical drainage monitoring system of claim 1, wherein the image processing circuit includes:
an image sampling module for sampling image frames from the video data; and
an image feature extraction module for extracting drainage data of the drained liquid from the sampled image frames by using a neural network.

15. The medical drainage monitoring system of claim 1, wherein the monitoring circuit includes:
a first data receiving unit for receiving drainage data from the image processing circuit;
a first data processing unit for comparing the drainage data with a threshold and triggering an alarm signal when the drainage data violates the threshold; and
a first alarm unit for generating an alarm in response to receiving the alarm signal from the first data processing unit.

16. The medical drainage monitoring system of claim 15, wherein the monitoring circuit further includes:
a first display unit for displaying the drainage data in real time.

17. The medical drainage monitoring system of claim 1, further comprising:
a central monitoring device configured to receive and monitor drainage data from a plurality of monitoring circuits.

18. The medical drainage monitoring system of claim 17, wherein the central monitoring device includes:
a second data receiving unit for receiving drainage data from a plurality of monitoring circuits;
a second data processing unit for comparing the drainage data with a threshold and triggering an alarm signal when the drainage data violates the threshold; and
a second alarm unit for generating an alarm in response to receiving the alarm signal from the second data processing unit.

19. The medical drainage monitoring system of claim 18, wherein the second alarm unit is further configured to generate an alarm in response to receiving the alarm signal from any one of the plurality of monitoring circuits.

20. The medical drainage monitoring system of claim 1, wherein the drainage container is a drainage bottle or a drainage bag.
